(19) United States
(12) Patent Application Publication
(10) Pub. No.: US 2012/0330035 A1
(43) Pub. Date: Dec. 27, 2012

(54) HYDROTHERMOLYSIS OF MONO- AND/OR OLIGOSACCHARIDES IN THE PRESENCE OF A POLYALKYLENE GLYCOL ETHER

(75) Inventors: Alois Kindler, Grunstadt (DE); Andrea Schmidt, Ludwigshafen (DE); Alwin Rehfinger, Mutterstadt (DE); Peter Bassler, Viernheim (DE); Ralf Böhling, Lorsch (DE)

(73) Assignee: BASF SE, Ludwigshafen (DE)

(21) Appl. No.: 13/530,890

(22) Filed: Jun. 22, 2012

Related U.S. Application Data
(60) Provisional application No. 61/500,637, filed on Jun. 24, 2011.

(51) Int. Cl. C07D 319/12; C07D 307/48; B01J 19/00

(52) U.S. Cl. 549/379; 422/162; 549/489

(57) ABSTRACT

The present invention relates to a method for the hydrothermolysis of a mono- and/or oligosaccharide-comprising composition which in addition comprises at least one monoalkyl and/or dialkyl ether of a polyalkylene glycol, and also relates to a hydrothermolysis device.

The present invention relates to a method for the hydrothermolysis of a mono- and/or oligosaccharide-comprising composition which in addition comprises at least one monoalkyl and/or dialkyl ether of a polyalkylene glycol, and also relates to a hydrothermolysis device.

Owing to the increasing security and rising prices of fossil raw materials, there is an increasing requirement for economically and ecologically acceptable alternatives. Methods which spare the resources by using renewable raw materials are increasing in importance in sustainable chemistry, especially in the field of polymer technology. In this sense, the reaction process revision comprises developing alternative synthetic pathways and also the use of alternative renewable raw materials, alternative solvents, alternative reactor types, and also alternative forms of energy input.

There is therefore a requirement for efficient methods for providing chemical fundamental materials and intermediates from the available biomass, e.g. from sugars. Valuable intermediates accessible from sugars and effectively useable are, e.g., hydroxyaldehydes, of which glycolaldehyde is the simplest member. Hydroxyaldehydes can be used, e.g., for producing polymers which comprise free hydroxyl groups, and also as intermediate for various esters and amino alcohols. Glycolaldehyde is used, for example, as starting material for α-amino acids, pharmaceuticals, agricultural chemicals, chemicals for photography, special polymers, fiber treatment agents and deodorants. An important intermediate is also 2,5-dihydroxy-1,4-dioxane (DHD) that is obtainable from glycolaldehyde by dimerization.

It is known to produce glycolaldehyde from ethylene glycol by catalytic oxydehydrogenation.

EP-A-0 217 280 describes a method for producing hydroxycarbonyl compounds by oxidative dehydrogenation of 1,2-diols in the gas phase using a catalyst made of hollow silver, copper or gold balls.

EP-A-0 376 182 describes a method for producing glycolaldehyde from ethylene glycol using a catalyst system that comprises copper or copper oxide and another metal or metal oxide. In the examples, a CuO/ZnO catalyst is used.

An alternative to the above described catalytic method is hydrothermolytic production from carbohydrates. Carbohydrates may be hydrothermolytically converted into polyalcohols and other industrially interesting compounds such as, for example, glycolaldehyde, hydroxymethylfurfural, DHD, etc.

In a research project of Dow Deutschland GmbH and the Fraunhofer Institut für Chemische Technologie, project numbers 22015600 and 22024200, the reductive (hydro)thermolysis of sugars is presented as a possible method for producing polyols from sugars. For this purpose low-molecular-weight carbohydrates such as glucose, fructose, xylose and sucrose, are converted to the desired short-chain polyalcohols by means of reductive hydrothermolysis in the presence of in-situ formed hydrogen as reducing agent. The best results were achieved using ruthenium as catalyst at temperatures between 150 and 250° C. The polyols thus obtained are described as suitable as starting material for producing polyurethanes.

WO 02/40436 discloses a spray pyrolysis in which an aqueous sugar solution is sprayed into a reactor and reacted at 500 to 600° C. to form a glycolaldehyde-comprising pyrolysis product.

In JP 2003104929, a method for producing glycolaldehyde from glucose is described, in which a retro-aldol condensation is carried out in supercritical water at 250 to 400 bar and 350 to 450° C. with a residence time of 0.02 to less than 1 second. For carrying out the reaction, preheated water is combined with an aqueous glucose solution that is at room temperature.

Kabyemela et al. in “Kinetics of Glucose Epimerization and Decomposition in Subcritical and Supercritical Water” (Ind. Eng. Chem. Res. 1997, 36, 1552-1558) study the decomposition of glucose in water at temperatures between 300° C. and 400° C., at a pressure of 250 to 400 bar and residence times of 0.02 to 2 seconds. A laboratory apparatus for carrying out the experiments is presented. Therein, water is preheated to a temperature of 15 K above the desired reaction temperature and, before entry into the reactor, is combined with an aqueous glucose solution which is at room temperature.

The two last-described hydrothermolytic methods have in common that the energy input proceeds in a direct manner, i.e. preheated water is mixed with a cold aqueous carbohydrate solution. The added water functions thereby as reaction partner and heat-transfer medium. Such a procedure is capable of further improvement. Owing to the mixing of streams having greatly differing temperature, viscosity and density, in a time period of a few milliseconds, droplets can form at the mixing point, the sugar can caramelize and it can lead to a blockage of the device. In addition to the susceptibilities to faults, the methods of the prior art are also energy- and cost-intensive. In particular methods, in which water is used under supercritical conditions, are not suitable for energy integration.

The object of the present invention is to provide an improved method for hydrothermolysis of mono- and/or oligosaccharides. Surprisingly, it has been found that this object is achieved when, for the hydrothermolysis, a monosaccharide- and/or oligosaccharide-comprising composition is used which, in addition, comprises at least one monoalkyl or dialkyl ether of a polyalkylene glycol. A further object of the present invention is to provide an improved hydrothermolysis device. Surprisingly, it has been found that this object is achieved by a hydrothermolysis device which comprises a heat-up zone having a hydraulic diameter of at most 3 mm.

The invention first relates to a continuous method for hydrothermolysis of a monosaccharide- and/or oligosaccharide-comprising composition, in which
i) a solution is provided which comprises at least one mono- and/or oligosaccharide, at least one monoalkyl or dialkyl ether of a polyalkylene glycol and water;
ii) the solution provided in step i) is heated abruptly in a heat-up zone;
iii) at least some of the at least one mono- and/or oligosaccharide present in the heated solution is hydrothermally reacted in a reaction zone; and
iv) the reaction mixture obtained in step iii) is quenched in a quench zone.

The invention further relates to a hydrothermolysis device, comprising
a heat-up zone;
a reaction zone;
a quench zone;
   wherein the heat-up zone has a hydraulic diameter of at most 3 mm, preferably at most 0.3 mm.

The invention further relates to a continuous method for hydrothermolysis of a monosaccharide- and/or oligosaccharide-comprising composition, in which
i) a solution is provided which comprises at least one mono- and/or oligosaccharide, at least one monoalkyl or dialkyl ether of a polyalkylene glycol and water;
ii) the solution provided in step i) is heated abruptly in a heat-up zone;
iii) at least some of the at least one mono- and/or oligosaccharide present in the heated solution is hydrothermally reacted in a reaction zone; and
iv) the reaction mixture obtained in step iii) is quenched in a quench zone,
   wherein the method is carried out in a hydrothermolysis device which comprises a heat-up zone which has a hydraulic diameter of at most 3 mm, particularly preferably at most 0.3 mm.

The method according to the invention is advantageous in embodiments thereof described hereinafter with respect to one or more of the following points:
the above described disadvantages of mixing starting material streams having highly differing temperature, viscosity and/or density before entry into the reaction zone are decreased or avoided;
especially, problems, such as droplet formation, caramelization of the sugar and/or blockages of the device, are avoided;
the method is more energy efficient and therefore cheaper than the methods known to date;
good selectivity, especially with respect to the dimerization products of the hydroxyaldehydes, such as DHD;
good yields;
continuous method;
the use of a catalyst is not absolutely necessary;
the device according to the invention is designed to be simple in terms of apparatus;
the abovementioned disadvantages, such as droplet formation, caramelization of the sugar and/or blockages can be avoided;
the device is maintenance-friendly and/or can be achieved with low capital costs.

Preferably, the aqueous solution provided in step i) is not mixed directly with a heat-transfer medium.

Preferably, neither for providing the solution in step i), nor for the abrupt heating in step ii), is the aqueous solution combined with superheated steam or supercritical water.

The method according to the invention makes possible the production of hydrothermolysis products of mono- and/or oligosaccharides, wherein the use of the customary catalysts known from the prior art for such hydrothermolysis reactions can be dispensed with. The monosaccharide- and/or oligosaccharide-comprising composition used for the hydrothermolysis is therefore not obligatorily additionally brought into contact with metals, such as Cu, Ag or Au, metal oxides, such as CuO/ZnO catalysts, or other catalysts.

Hydrothermolysis in the context of the present invention is taken to mean a thermal reaction in the presence of H₂O. Here and hereinafter it is also termed a hydrothermal reaction. According to the invention, the hydrothermal reaction proceeds in a medium which comprises at least one monoalkyl or dialkyl ether of a polyalkylene glycol and water.

### Step i)

### Mono- and Oligosaccharides

According to the invention, in step i), a solution is provided which comprises at least one mono- and/or oligosaccharide.

The term “oligosaccharides” designates preferably carbohydrates which have 2 to 6 monosaccharide units.

Preferably, the solution provided in step i) has a content of mono- and/or oligosaccharides in the range from 0.1 to 50% by weight, particularly preferably in the range from 0.5 to 20% by weight, in particular 1 to 15% by weight, based on the total weight of the composition.

Preferably, mono- and/or oligosaccharides present in the solution provided in step i) comprise at least 95% by weight, preferably at least 99% by weight, based on the total weight of the mono- and/or oligosaccharides, of mono- and/or disaccharides.

Preferably, the mono- and/or oligosaccharides present in the solution provided in step i) are selected from
aldopentoses,
aldohexoses,
ketohexoses,
disaccharides that are derived from aldopentoses, aldohexoses, ketohexoses and mixtures thereof, and also
mixtures thereof.

Particularly preferably, the mono- and/or oligosaccharides present in the solution provided in step i) are selected from glucose, xylose, fructose, sucrose and mixtures thereof.

### Mono- or Dialkyl Ethers of a Polyalkylene Glycol

Preferably, the solution provided in step i) has a content of mono- and/or dialkyl ether of a polyalkylene glycol in the range from 15 to 99% by weight, particularly preferably in the range from 20 to 95% by weight, based on the total weight of the solution.

The mono- or dialkyl ethers used according to the invention of a polyalkylene glycol are preferably selected from mono(C₁-C₆ alkyl)ethers of a poly(C₁-C₆-alkylene)glycol, di(C₁-C₆-alkyl)ethers of a poly(C₁-C₆-alkylene)glycol, and mixtures thereof.

Particularly preferably, the monoalkyl or dialkyl ethers of a polyalkylene glycol are selected from
ethylene glycol monomethyl ether,
ethylene glycol monoethyl ether,
ethylene glycol monopropyl ether,
ethylene glycol monoisopropyl ether,
ethylene glycol monobutyl ether,
1,2-propanediol 1-monomethyl ether,
1,2-propanediol 2-monomethyl ether,
1,3-propanediol monomethyl ether,
1,2-propanediol 1-monoethyl ether,
1,2-propanediol 2-monoethyl ether,
1,3-propanediol monoethyl ether,
1,2-propanediol dimethyl ether,
1,3-propanediol dimethyl ether,
1,2-propanediol diethyl ether,
1,3-propanediol diethyl ether,
diethylene glycol monomethyl ether,
diethylene glycol monoethyl ether,
diethylene glycol mono(n-butyl)ether,
ethylene glycol dimethyl ether,
ethylene glycol diethyl ether,
ethylene glycol dibutyl ether,
diethylene glycol dimethyl ether,
diethylene glycol diethyl ether,
diethylene glycol di(n-butyl)ether,
triethylene glycol dimethyl ether,
triethylene glycol diethyl ether,
triethylene glycol di(n-butyl)ether,
tetraethylene glycol dimethyl ether,
tetraethylene glycol diethyl ether,
tetraethylene glycol di(n-butyl)ether,
   mixtures of two or more of the abovementioned compounds.

In particular, the monoalkyl or dialkyl ethers of a polyalkylene glycol are selected from ethylene glycol dimethyl ether, diethylene glycol dimethyl ether, triethylene glycol dimethyl ether and tetraethylene glycol dimethyl ether.

Preferably, the solution provided in step i) has a water content in the range from 0.5 to 65% by weight, particularly preferably in the range from 1 to 30% by weight, based on the total weight of the solution.

If desired, the solution provided in step i) can have at least one organic water-miscible solvent different from monoalkyl and dialkyl ethers of polyalkylene glycol.

Preferably, the solution provided in step i) has a content of organic, water-miscible solvents in the range from 0 to 55% by weight, particularly preferably in the range from 0.1 to 25% by weight, in particular from 0.5 to 10% by weight, based on the total weight of the solution.

Examples of water-miscible organic solvents are C₁-C₄ alkanols such as methanol, ethanol, n-propanol, isopropanol, n-butanol, ethers such as diethyl ether, cyclic ethers such as dioxane and tetrahydrofuran, and also alkylene carbonates such as ethylene carbonate (2-oxo-1,3-dioxolane) and propylene carbonate (2-oxo-1,3-dioxane).

A special solution provided in step i) comprises
5 to 55% by weight water,
50 to 94.5% by weight of a monoalkyl or dialkyl ether of a polyalkylene glycol selected from ethylene glycol dimethyl ether, diethylene glycol dimethyl ether, triethylene glycol dimethyl ether and tetraethylene glycol dimethyl ether,
0.5 to 45% by weight of glucose.

A preferred embodiment of the method according to the invention is hydrothermolysis of glucose for producing 2,5-dihydroxy-1,4-dioxane (DHD).

A preferred embodiment of the method according to the invention is hydrothermolysis of sucrose for producing 2,5-dihydroxy-1,4-dioxane (DHD).

A preferred embodiment of the method according to the invention is hydrothermolysis of xylose for producing furfural.

A preferred embodiment of the method according to the invention is hydrothermolysis of fructose for producing 5-hydroxymethylfurfural (5-HMF).

### Step ii)

A heat-up zone, in the context of the present invention, is taken to mean the section of a heat exchanger in the direction of flow of the mass stream/mass streams in which heating takes place. The heat-up zone can be arranged within a part of a heat exchanger, within an entire heat exchanger, or within two or more heat exchangers.

Abrupt heating, in the context of the present invention, is intended to mean that the heating of a medium from the temperature predetermined by the surroundings of the medium to the required reaction temperature takes at most one minute.

In a preferred embodiment of the method according to the invention, the heating in step ii) proceeds with a residence time in the heat-up zone in the range from 1 ms to 1 s.

In particular, the heating in step ii) proceeds at a heating rate β≧30 K/s, particularly preferably ≧300 K/s, in particular ≧1000 K/s, especially ≧10 000 K/s.

The heating rate β is defined as follows:

β=ΔT_{H}/Δt_{H}

where
β is the heating rate [K/s]
ΔT_{H} is the temperature interval [K] passed through for heating up
Δt_{H} is the time interval [s] required for heating up

In a preferred embodiment of the method according to the invention, the steps ii) and

iii) are carried out at a pressure in the range from 100 bar to 400 bar, preferably in the range from 200 bar to 300 bar.

Step iii)

A reaction zone in the context of the present invention is taken to mean the section of a reactor in the direction of flow of the material stream/material streams in which a chemical reaction proceeds. The reaction zone can be arranged within a part of a reactor, within a total reactor, or within two or more reactors.

Preferably, in step iii), the temperature in the reaction zone is carried out in a range from 150° C. to 500° C., particularly preferably from 180° C. to 400° C., particularly preferably in a range from 200° C. to 350° C.

In a suitable embodiment of the method according to the invention, the water present in the heated solution in step iii) is in the supercritical state.

In the context of the present invention, the supercritical state shall be taken to mean the thermodynamic state of a substance which is characterized by the densities of liquid phase and gas phase being equal. The differences between both states of matter cease to exist here. In the phase diagram, the critical point is the upper end of the vapor pressure curve. Since, above the critical point, liquid and gas can no longer be differentiated from one another, a supercritical fluid or a supercritical state are spoken of. Supercritical water combines the high dissolution capacity of liquid water with the low viscosity similar to that of (gaseous) water vapor.

In an alternative embodiment of the method according to the invention, the water present in the heated solution in step iii) is not in the supercritical state. The method according to the invention, in which a mono- and/or oligosaccharide-comprising composition is used for the hydrothermolysis which comprises, in addition to water, at least one monoalkyl or dialkyl ether of a polyalkylene glycol, permits an advantageous procedure without the water in the reaction zone being in the supercritical state. This permits a markedly more energy efficient procedure.

In a preferred embodiment of the method according to the invention, the step iii) is passed through with a residence time in the range from 0.1 s to 120 s.

In a further preferred embodiment of the method according to the invention, the ratio of the residence time in the heat-up zone to the residence time in the reaction zone is in the range from 1:10 to 1:10⁴.

### Step iv)

Quenching is taken to mean quite in general the rapid stopping of a proceeding reaction. In the context of the present invention, it is taken to mean that the reaction mixture is cooled so intensely that further reaction, for example leading to unwanted secondary products, is prevented.

In a particularly preferred embodiment of the method according to the invention, during quenching in step iv), the temperature interval ΔT_{K} between reaction temperature T_{R} and T≦120° C. is passed through within a time interval Δt_{K}≦1 s, especially Δt_{K}≦0.1 s. In this case ΔT_{K} denotes the temperature interval passed through for cooling, and Δt_{K} denotes the time interval required for cooling.

In an equally preferred embodiment of the method according to the invention, the ratio of the residence time in the heat-up zone to the residence time in the reaction zone is in the range from 1:10 to 1:10⁴.

In particular, the method according to the invention is suitable for producing
dihydroxydioxane from glucose, or
dihydroxydioxane from sucrose, or
furfural from xylose, or
5-hydroxymethylfurfural from fructose.

In a suitable embodiment of the method according to the invention, energy integration between steps iii) and iv) is provided.

Energy integration can increase the energy efficiency of the overall process. In particular, the steps ii) and iv) according to the invention may be thermally connected to one another in an advantageous manner.

Pinch analysis provides a possible approach for the systematic optimization of the energy consumption of the process. Pinch analysis is a method for minimizing the energy consumption of engineering processes in which thermodynamically minimum energy consumptions are calculated. This method also indicates how these minimum energy consumptions can be achieved, for example by matching heat transfer networks for heat recovery, energy supply and process conditions to one another. Pinch analysis was and is frequently dealt with in the specialist literature, and is also available as software and is familiar to those skilled in the art.

The present invention further relates to a hydrothermolysis device which comprises
a heat-up zone;
a reaction zone;
a quench zone.

The heat-up zone, here and hereinafter, is taken to mean the section of the device according to the invention in the direction of flow of the starting material stream/streams in which the abrupt heating takes place. The heat-up zone can be arranged within a part of a heat exchanger, within an entire heat exchanger, or within two or more heat exchangers.

The reaction zone, here and hereinafter, is taken to mean the section of the device according to the invention in the direction of flow of the starting material stream/streams in which the hydrothermal reaction proceeds. The starting material stream/streams enter into the reaction zone and leave it as a product stream. The reaction zone can be arranged within a part of a reactor, within an overall reactor, or within two or more reactors.

The quench zone, here and hereinafter, is taken to mean the section of the device according to the invention in the direction of flow of the product stream in which the quenching takes place. The quench zone can be arranged within a part of a heat exchanger, within an entire heat exchanger, or within two or more heat exchangers.

In a suitable embodiment, two or all three of the zones heat-up zone, reaction zone and quench zone are not constructed so as to be structurally separated from one another. This includes, e.g. a structurally uniform construction of two or three of the zones in one tube which is successively conducted through two or more heat exchangers, where in the direction of flow of the reaction medium, first forming the heat-up zone, heat is supplied and the reaction initiated, and then, for forming the quench zone, heat is taken off again. The terms heat-up zone, reaction zone and quench zone are then to be understood functionally and not structurally. Of course, in the heat-up zone, after the reaction temperature is reached, also, a hydrothermal reaction already proceeds. Via integration of two or all three of the zones—heat-up zone, reaction zone and quench zone—into a structural unit, the complexity in terms of apparatus may be minimized.

The heat-up zone has according to the invention a hydraulic diameter of at most 3 mm, preferably at most 0.3 mm.

The hydraulic diameter dₕ is a theoretical quantity for carrying out studies and calculations on tubes or channels having non-circular cross section and comparison with circular cross sections. Using the hydraulic diameter dₕ, then, as with the internal diameter of a round tube, calculations can be performed. It is the quotient of four times the flow cross-sectional area A and the wetted perimeter U:

dₕ=4*A/U

where
dₕ is the hydraulic diameter,
A is the flow cross-sectional area,
U is the wetted perimeter.

In a preferred embodiment of the hydrothermolysis device according to the invention, the reaction zone has a hydraulic diameter of not greater than three times the hydraulic diameter of the heat-up zone.

The hydraulic diameter of the reaction zone is according to the invention at most 10 mm, e.g. 0.01 to 10 mm, or preferably 0.02 to 10 mm, or particularly preferably 0.05 to 10 mm; preferably at most 3 mm, e.g. 0.01 to 3 mm, or preferably 0.02 to 3 mm, or particularly preferably 0.05 to 3 mm.

In an equally preferred embodiment of the hydrothermolysis device according to the invention, at least one of the three zones has microstructures.

Conventional installations and installations having microstructures differ by their characteristic dimension. Conventional installations have a characteristic dimension of >10 mm, microstructured installations in contrast of ≦10 mm.

The characteristic dimension of an installation, e.g. a heat-up zone, a reaction zone or a quench zone, is taken to mean, in the context of the present installation, the hydraulic diameter. The hydraulic diameter of an installation having microstructures, here and hereinafter and also termed a microstructured installation, is markedly smaller than that of a conventional reactor (e.g. by at least the factor 10, or at least the factor 100, or at least by the factor 1000) and is customarily in the range from one hundred nanometers to ten millimeters. Frequently, the hydraulic diameter is in the range from 1 μm to 1 mm. In comparison with customary reactors, therefore, microstructured installations exhibit a significantly different behavior with respect to the heat and mass transport processes proceeding. Owing to the greater ratio of surface area to reactor volume, for example, a very good heat supply and removal is made possible, for which reason, for example, even highly endo- or exothermic reactions proceed virtually isothermally or large temperature regions may be passed through in very short time intervals.

Channels having a hydraulic diameter of less than 1 mm are described as microchannels. Microfluidics is concerned with the handling of liquids and gases in the smallest possible space, wherein the fluids are moved, mixed, separated or processed in other ways. Depending on the fluid (liquid or gas), for such flows, in addition to the customary effects (inertia, pressure, viscosity), other effects can occur which may be ignored in macroscopic flows. These effects can be of importance for optimizing a process, such as, for example, heat and mass transport in such small channels. Suitable installations and useable structures thereof are known in principle from the macro world and most can be scaled, taking into account specific parameters for small dimensions. Examples of installations having microchannels are, for example, microheat exchangers, micromixers, microreactors and mixed forms.

A particularly preferred embodiment of the hydrothermolysis device according to the invention comprises
a) a receiver vessel in which an aqueous solution is provided which comprises at least one mono- and/or oligosaccharide;
b) a heat-up zone in which the aqueous solution is heated abruptly;
c) a reaction zone in which the mono- and/or oligosaccharides present in the aqueous solution are partially or completely hydrothermally reacted;
d) a quench zone in which the reaction mixture is cooled to a temperature below 120° C. in the course of at most 0.1 minute;
e) a pressure expansion in which the reaction mixture is expanded to ambient pressure;
f) a discharge vessel in which the resultant reaction mixture is collected.

In a suitable embodiment of the hydrothermolysis device according to the invention, the heat-up zone b) comprises an externally heated tube. In a special embodiment, the heated tube has an internal diameter in the range from 20 μm to 2 mm, preferably from 100 μm to 500 μm. In particular, the heated tube has a ratio of tube length to internal diameter of 10² to 10⁷.

In an equally suitable embodiment of the hydrothermolysis device according to the invention, the heat-up zone b) comprises a channel in a microstructured apparatus. In particular, the channel has a ratio of length to internal diameter of 10² to 10⁷.

Micro heat exchangers that are to be used according to the invention for the heat-up zone are preferably selected from temperature-controllable tubes, tube-bundle heat exchangers, plate heat exchangers and temperature-controllable tubular reactors having internals. Tubes and tube-bundle heat exchangers to be used according to the invention, as characteristic dimensions, have tube or capillary internal diameters in the range preferably from 0.01 mm to 3 mm, particularly preferably in the range from 0.02 mm to 2 mm, and in particular in the range from 0.1 to 0.5 mm. Plate heat exchangers to be used according to the invention have layer heights or channel widths in the range of preferably 0.02 mm to 10 mm, particularly preferably in the range from 0.02 mm to 6 mm, and in particular in the range from 0.02 mm to 4 mm. Tubular reactors to be used according to the invention that have internals have tube diameters in the range from 0.5 mm to 100 mm, preferably in the range from 1 mm to 50 mm, and particularly preferably in the range from 1 mm to 20 mm. Alternatively, flat channels having inserted mixed structures comparable to plate apparatuses can also be used according to the invention. They have heights in the range from 0.1 mm to 20 mm and widths in the range from 1 mm to 100 mm, and in particular in the range from 1 mm to 50 mm. Optionally, the tubular reactors can comprise mixing elements through which pass temperature-controlled channels (such as, e.g., CSE-XR® type from Fluitec, CH).

Micro reaction zones to be used according to the invention are preferably selected from optionally temperature-controllable tubular reactors, tube bundles and optionally temperature-controllable tubular reactors having internals. Tubular reactors and tube bundles to be used according to the invention, as characteristic dimensions, have tube or capillary diameters in the range from preferably 0.01 mm to 10 mm, particularly preferably in the range from 0.02 mm to 9 mm, more preferably in the range from 0.03 mm to 3 mm, and in particular in the range from 0.05 mm to 1 mm. Tubular reactors having internals to be used according to the invention have tubular diameters in the range from 0.5 mm to 250 mm, preferably in the range from 1 mm to 100 mm, and particularly preferably in the range from 1 mm to 50 mm. Alternatively, flat channels having inserted mixing structures which are comparable to plate apparatuses can also be used according to the invention. They have heights in the range from 0.1 mm to 20 mm and widths in the range from 1 mm to 100 mm, and in particular in the range from 1 mm to 50 mm. Optionally, the tubular reactors can comprise mixing elements, through which pass temperature-controlled channels (such as, e.g., the CSE-XR® type from Fluitec, CH).

For the micro heat exchangers to be used according to the invention for the quench zone, that stated above applies with respect to the micro heat exchangers for the heat-up zone.

The optimum characteristic dimension results in this case from the requirements for the permissible anisothermy of the reaction profile, the maximum permissible pressure drop and the susceptibility to blockage of the reactor.

Particularly preferred micro heat exchangers are:
tubular reactors made of capillaries or capillary bundles having tubular cross sections from 20 μm to 2 mm, preferably from 50 μm to 1 mm, particularly preferably from 100 μm to 500 μm, with or without additional mixing internals, wherein the tubes or capillaries can be flushed by a temperature-control medium;
tubular reactors in which the heat carrier is conducted into the capillaries/tubes, and the product which is to be temperature-controlled is conducted around the tubes and is homogenized by internals (mixing elements), such as, for example, of the CSE-SX® type from Fluitec, CH;
plate reactors which are constructed like plate heat exchangers having insulated parallel channels, networks of channels or surfaces which are equipped with or without flow-baffling internals (studs), wherein the plates conduct product and heat carrier in parallel or in a layer structure which has heat carrier and product layers alternately, such that during the reaction the chemical and thermal homogeneity can be insured;
and also
reactors having “flat” channel structures which have a “microdimension” only in the height and can be virtually as wide as desired, the typical comb-like internals of which prevent the formation of a flow profile and lead to a narrow residence time distribution important for the defined reaction procedure and residence time.

Particularly preferred microreactors are:
tubular reactors made of capillaries or capillary bundles having tubular cross sections from 20 μm to 5 mm, preferably from 50 μm to 3 mm, particularly preferably from 100 μm to 1 mm, with or without additional mixing internals, wherein the tubes or capillaries can optionally be flushed by a temperature-control medium.

In a preferred embodiment of the invention, a microstructured reaction zone is used for the hydrothermal reaction. The microstructured reaction zone, here and hereinafter, is also termed as a reaction zone having microstructures or microreaction zone. Microstructured reaction zones are suitable for ensuring the thermal homogeneity perpendicular to the direction of flow. In this case, in principle, each differential volume element has substantially the same temperature over the respective flow cross section. The maximum permissible temperature differences in a flow cross section depend in this case on the desired product properties. Preferably, the maximum temperature difference in a flow cross section is less than 50° C., particularly preferably less than 20° C., and in particular less than 10° C.

In a very particularly preferred embodiment of the invention, a microreaction zone is used which has the residence time characteristics of a plug flow. If a plug flow is present in a tube (reactor), the state of the reaction mixture (e.g. temperature, composition etc.) can thus vary in the flow direction; in contrast, for each individual cross section, perpendicular to the flow direction, the state of the reaction mixture is the same. Therefore, all of the volume elements entering into the tube have the same residence time in the reactor. Depicted visually, the liquid flows through the tube as if it were a sequence of plugs readily sliding through the tube. In addition, crossmixing due to the intensified mass transport perpendicular to the direction of flow can harmonize the concentration gradient perpendicular to the direction of flow.

Therefore, backmixing may be avoided, despite the usually laminar flow through apparatuses having microstructures, and a narrow residence time distribution can be achieved similar to that in an ideal flow tube.

The Bodenstein number Bo is a dimensionless quantity and describes the ratio of the convection stream to the dispersion stream (e.g. M. Baerns, H. Hofmann, A. Renken, Chemische Reaktionstechnik, Lehrbuch der Technischen Chemie [Chemical Reaction Technology, Handbook of Industrial Chemistry], Volume 1, 2nd Edition, pp. 332 ff). It therefore characterizes the backmixing within a system:

$$Bo = \frac{uL}{{D}_{ax}}$$

where
Bo is the Bodenstein number [−]
u is the flow velocity [ms⁻¹]
L is the length of the reactor [m]
Dₐₓ is the axial dispersion coefficient [m²h⁻¹].

A Bodenstein number of zero corresponds to complete backmixing in an ideal continuous stirred tank. An infinitely large Bodenstein number, in contrast, means absolutely no backmixing, as is the case in continuous flow through an ideal flow tube.

In capillaries (capillary reactors), the desired backmixing behavior can be set by setting the ratio of length to diameter in dependence on the material parameters and the flow state. The underlying calculation rules are known to those skilled in the art (e.g. M. Baerns, H. Hofmann, A. Renken: Chemische Reaktionstechnik, Lehrbuch der Technischen Chemie, Volume 1, 2nd Edition, pp. 339 ff). If a backmixing behavior as low as possible is to be achieved, the above defined Bodenstein number is selected to be preferably greater than 10, particularly preferably greater than 20, and in particular greater than 50. For a Bodenstein number greater than 100, the capillary reactor then substantially has a plug-flow character.

Suitable materials for the devices to be used according to the invention and/or as corresponding coatings of these devices are the materials known to those skilled in the art for use in a range of high temperatures and high pressures. These include austenitic stainless steels, such as 1.4541 or 1.4571, generally known as V4A or as V2A, respectively, and stainless steels of the US types SS316 and SS317Ti. Those which are likewise suitable are high-temperature-resistant thermoplastics, such as polyaryl ether ketones (PAEK) and especially polyether ether ketones (PEEK). Likewise suitable are Hastelloy® types, glass or ceramics. Suitable materials are, in addition, TiN₃, Ni-PTFE, Ni-PFA or the like.

In a special embodiment of the hydrothermolysis device according to the invention, the heat-up zone b) and the quench zone d) are arranged such that energy integration between b) and d) can be utilized.

The starting material stream in the form of an aqueous solution can be heated by the hot product stream leaving the reaction zone in direct exchange or by means of a temperature-control medium. If the energy integration between the heat-up zone and the quench zone proceeds in direct exchange, the heat-up zone and the quench zone are combined, for example, in one structural unit. For this purpose, the heat-up zone and the quench zone are arranged in a heat exchanger in such a manner that the product stream that is to be cooled takes over the function of the heat carrier, whereas the starting material stream that is to be heated removes the heat transferred from the product stream. In this case the two streams can be conducted in cocurrent flow, in cross flow or in countercurrent flow.

Optionally, the energy integration can also proceed via a temperature-control medium. For this purpose, the product stream is cooled in a heat exchanger by means of a suitable heat transfer medium and the starting material stream is heated in a further heat exchanger by means of the hot heat transfer medium.

Preferably, the energy integration proceeds in direct exchange in a suitable heat exchanger in such a manner that the starting material stream and the product stream are conducted in countercurrent flow.

If the starting material stream does not achieve the required reaction temperature during the heat-up in b), an additional heat exchanger needs to be provided within, or as part of, the heat-up zone.

If the product stream does not reach the required quench temperature of below 120° C. during cooling in d), an additional heat exchanger needs to be provided within, or as part of, the quench zone.

By means of pinch analysis, a person skilled in the art can calculate a corresponding energy integration and design the required heat exchangers or heat exchange networks for heat recovery.

In a particularly preferred embodiment, the method according to the invention is carried out in a hydrothermolysis device according to the invention.

The method according to the invention and the device according to the invention offer a number of possible advantages compared with the prior art, not only with respect to economic efficiency, but also with respect to environmental acceptability:

### Economic Efficiency

optimized energy consumption and mass consumption by circulation processes
simple plant structure and universality in use
utilization of untapped potentials of biogenic mass (residues and waste materials from agriculture or the food sector)

### Environmental Acceptability

obtaining the (intermediate) product from renewable raw materials
water as solvent
additionally used solvents or additives can be recovered and/or circulated
reduction of waste streams by material utilization of biomass

The method according to the invention and the device according to the invention will be described in more detail hereinafter with reference to the figures, without limiting the invention thereto.

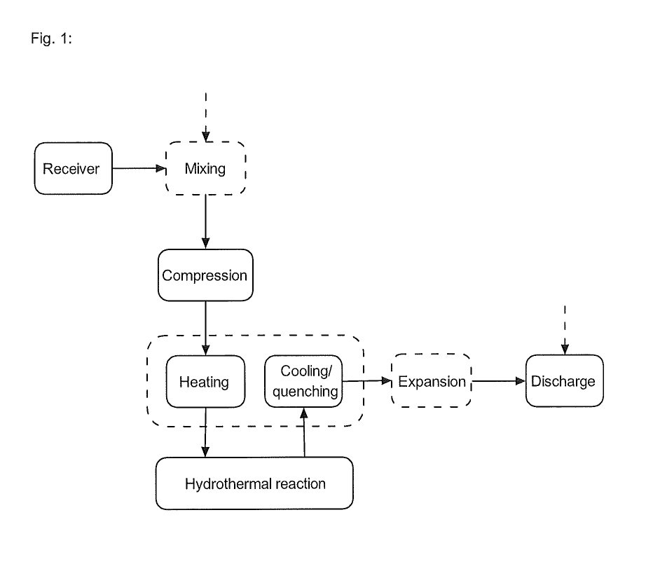

FIG. 1 shows a block diagram of the method according to the invention.

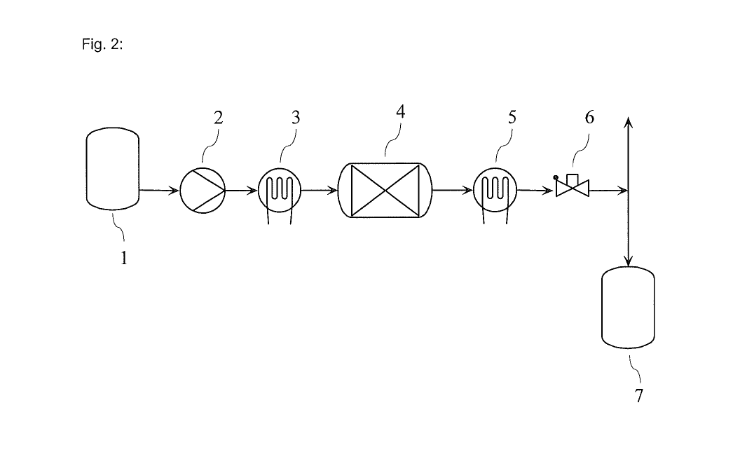

FIG. 2 shows the schematic structure of a device according to the invention.

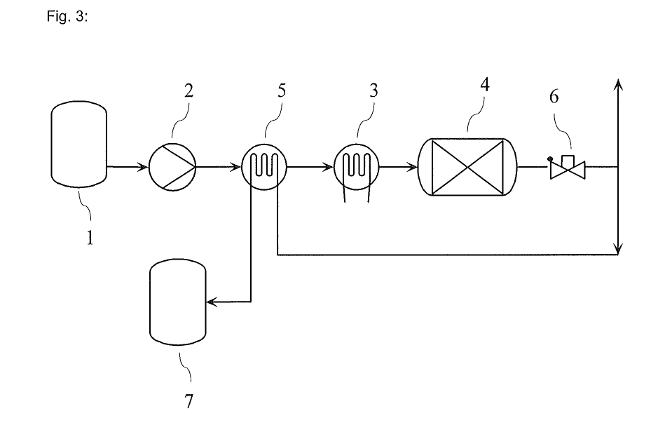

FIG. 3 shows the schematic structure of a device according to the invention with energy integration.

The method according to the invention will be described with reference to the block diagram shown in FIG. 1. Lines or blocks shown dashed are optional steps.

In a receiver, a solution is provided that contains at least one mono- and/or oligosaccharide and a solvent. In this case the solvent used can be water, at least one mono- or dialkyl ether or a polyalkylene glycol, or a mixture thereof. The receiver can comprise directly all components in the desired amounts to be used. Alternatively, a solvent component that is not present in the receiver can be added in a separate step “mixing”. It is also possible to add additional water and/or additional polyalkylene glycol ether in a separate step “mixing”. Likewise, if wished, a further organic, water-miscible solvent can be provided directly in the receiver, or added in a separate step “mixing”. The mixing can proceed either in the receiver or else in another suitable appliance. Preferably, for providing the mono- and/or oligosaccharide-comprising solution, the receiver is not mixed with a component which has a temperature markedly higher than the receiver. In particular, the receiver is not mixed with superheated steam or supercritical water.

The solution provided or the mixed solution is then compressed. The compression proceeds preferably to at least 100 bar. The compression proceeds suitably via a pump, in particular via a piston pump.

The compressed solution is abruptly heated. The solution is heated preferably with a residence time of at most one minute. For this purpose, preferably a high heating rate, as defined above, is achieved.

The hydrothermal reaction of the saccharide present in the solution proceeds at a temperature in the range from 150° C. to 500° C. The residence time for this step is preferably in the range of some tenths of a second up to three minutes. The residence time for the hydrothermal reaction is preferably up to four orders of magnitude higher than the residence time for the heating.

Immediately following the hydrothermal reaction, quenching proceeds, in order to stop the reaction and to prevent further reaction to form unwanted secondary products. During the quenching, the reaction mixture is cooled within a very short time to a temperature below the reaction temperature. The residence time for the hydrothermal reaction is likewise higher by up to four orders of magnitude than the residence time during the quenching.

The reaction mixture is then expanded to ambient pressure. The expansion can proceed together with the quenching or in a separate step. The expansion can proceed in the quench zone or in another suitable appliance. The reaction mixture is finally collected as discharge in a suitable appliance and optionally subjected to further processing and/or admixed with additives. In a special embodiment, the discharge is subjected to separation by distillation for obtaining the product of value.

In a suitable manner, the steps heating and quenching can be heat-connected. By means of such energy integration between these steps, the energy efficiency of the overall process is significantly increased. For this purpose, e.g., the heat recovered during quenching can be transferred to a heat-transfer medium and this can be used in the heating of the reaction starting mixture. Alternatively, the reaction discharge can also be conducted directly through a heat exchanger which is used in the heating of the reaction starting mixture. It is also possible to use the heat recovered in the purification by distillation of the reaction product.

FIGS. 2 and 3 show the schematic structure of a device according to the invention. In FIGS. 2 and 3 the following reference signs are used:
1 receiver vessel saccharide/polyalkylene glycol ether/water
2 (piston) pump
3 heat exchanger
4 capillary tube reactor
5 heat exchanger optionally with energy integration
6 pressure expansion valve
7 discharge or product vessel

With reference to FIG. 2, the structure of a device according to the invention without energy integration will be described hereinafter: in a receiver vessel 1, a saccharide solution is provided which comprises the mono- and/or oligosaccharide to be reacted in a mixture of a monoalkyl or dialkyl ether of a polyalkylene glycol and water. From the receiver vessel 1, by means of a piston pump 2, the saccharide solution is metered and compressed. The aqueous solution enters at a pressure of at least 100 bar into a heat exchanger 3. In the heat exchanger 3, the aqueous solution is abruptly, i.e. with a residence time of less than one minute, heated to the reaction temperature. The solution heated to reaction temperature is further passed into a capillary tube reactor 4. In the capillary tube reactor 4, the saccharide present in the solution is subjected to a hydrothermal reaction at a temperature in the range from 150° C. to 500° C. From the capillary tube reactor 4, the reaction mixture passes directly into the heat exchanger 5. In the heat exchanger 5, the reaction mixture is subjected to an intensive quench and is cooled in the course of at most one minute to below 120° C. Via the pressure expansion valve 6, the reaction mixture is expanded to ambient pressure and passed into a discharge or product vessel 7. At this point it is possible, for example via a gas connection tube and/or a takeoff, to withdraw gaseous components from the reaction mixture.

With reference to FIG. 3, the structure of a device according to the invention with energy integration is described: in a receiver vessel 1, a saccharide solution is provided that comprises the mono- and/or oligosaccharide to be reacted in a mixture of a monoalkyl or dialkyl ether of a polyalkylene glycol and water. From the receiver vessel 1, by means of a piston pump 2, the saccharide solution is metered and compressed. The aqueous solution enters into a heat exchanger 5 at a pressure of at least 100 bar. In the heat exchanger 5, the aqueous solution is abruptly heated, wherein a heat transfer from the hot product stream to the aqueous solution takes place. If the aqueous solution in this case is not heated to the required reaction temperature, a second heat exchanger 3 is connected downstream, in which the aqueous solution is then heated to the reaction temperature. The residence time in the heat exchanger 5 and optionally heat exchanger 3 together does not exceed one minute. The solution heated to reaction temperature is further passed into a capillary tube reactor 4. In the capillary tube reactor 4, the saccharide present in the solution is subjected to a hydrothermal reaction at a temperature in the range from 150° C. to 500° C. Via the pressure expansion valve 6, the reaction mixture is expanded. At this point it is possible, for example via a gas collection tube and/or a takeoff, to withdraw gaseous components from the reaction mixture. The hot reaction mixture is further passed into the heat exchanger 5. In the heat exchanger 5, the reaction mixture is cooled to below 120° C. within at mostone minute. For this purpose, the heat-up zone and the quench zone are arranged in the heat exchanger 5 in such a manner that the product stream that is to be cooled takes over the function of the heat carrier, whereas the feed stream that is to be heated removes the heat transferred from the product stream. The two streams are advantageously conducted in cross-flow or counter-current flow in the heat exchanger 5. The cooled product stream is then passed into a discharge vessel or product vessel 7.

In order to achieve the short residence times in the device according to the invention, and in particular in the heat-up zone and quench zone, preferably, not only the heat exchangers 5 and 7, but also the capillary tube reactor 6, have microchannels or microstructures.

The microchannels in the heat exchangers 5 and 7 advantageously have about the same inner diameter of not more than 0.3 mm, whereas the inner diameter of the capillary tube(s) in the reactor 6 does not exceed a value of 1 mm.

1.-33. (canceled)

34. A continuous method for hydrothermolysis of a monosaccharide- and/or oligosaccharide-comprising composition, comprising:
i) providing a solution which comprises at least one mono- and/or oligosaccharide, at least one monoalkyl or dialkyl ether of a polyalkylene glycol and water;
ii) heating the solution provided in step i) abruptly in a heat-up zone;
iii) hydrothermally reacting at least some of the at least one mono- and/or oligosaccharide present in the heated solution in a reaction zone to obtain a reaction mixture; and
iv) quenching the reaction mixture obtained in step iii) in a quench zone.

35. The method according to claim 34, wherein the solution provided in step i) comprises the at least one monoalkyl or dialkyl ether of a polyalkylene glycol in an amount from 15 to 99% by weight, based on the total weight of the solution.

36. The method according to claim 34, wherein the solution provided in step i) has a water content in the range from 0.5 to 65% by weight, based on the total weight of the solution.

37. The method according to claim 34, wherein the solution provided in step i) has a content of the at least one mono- and/or oligosaccharide in the range from 0.1 to 50% by weight, based on the total weight of the solution.

38. The method according to claim 34, wherein the solution provided in step i) has a content of the at least one mono- and/or oligosaccharide in the range from 1 to 15% by weight, based on the total weight of the solution; a water content in the range from 1 to 30% by weight, based on the total weight of the solution; and comprises the at least one monoalkyl or dialkyl ether of a polyalkylene glycol in an amount from 20 to 95% by weight, based on the total weight of the solution.

39. The method according to claim 34, wherein the solution provided in step i) comprises
5 to 55% by weight water,
50 to 94.5% by weight of a monoalkyl or dialkyl ether of a polyalkylene glycol selected from ethylene glycol dimethyl ether, diethylene glycol dimethyl ether, triethylene glycol dimethyl ether and tetraethylene glycol dimethyl ether,
0.5 to 45% by weight of glucose.

40. The method according to claim 34, wherein the monoalkyl or dialkyl ether of a polyalkylene glycol used in step i) is selected from C₁-C₆ monoalkylene glycols etherified on one side or both sides with a C₁-C₆ alkanol and C₁-C₆ polyalkylene glycols etherified on one side or both sides with a C₁-C₆ alkanol.

41. The method according to claim 34, wherein the at least one mono- and/or oligosaccharide present in the solution provided in step i) is selected from glucose, xylose, fructose, sucrose and mixtures thereof.

42. The method according to claim 34, wherein the heating in step ii) proceeds with a residence time in the heat-up zone in the range from 1 ms to 1 s.

43. The method according to claim 34, wherein the heating in step ii) proceeds at a heating rate β=ΔT_{H}/Δt_{H}≧30 K/s.

44. The method according to claim 34, wherein the steps ii) and iii) are carried out at a pressure in the range from 100 bar to 400 bar.

45. The method according to claim 34, wherein, in step iii), the temperature in the reaction zone is in a range from 150° C. to 500° C.

46. The method according to claim 34, wherein the heating in step ii) proceeds at a heating rate β=ΔT_{H}/Δt_{H}≧300 K/s, steps ii) and iii) are carried out at a pressure in the range from 200 bar to 300 bar, and in step iii), the temperature in the reaction zone is in a range from 180° C. to 400° C.

47. The method according to claim 34, wherein the step iii) is passed through with a residence time in the range from 0.1 s to 120 s.

48. The method according to claim 34, wherein the ratio of the residence time in the heat-up zone to the residence time in the reaction zone is in the range from 1:10 to 1:10⁴.

49. The method according to claim 34, wherein the heat-up zone has a ratio of length to internal diameter of 5:1 to 5000:1.

50. The method according to claim 34, wherein the reaction zone has an internal diameter of not more than three times the internal diameter of the heat-up zone.

51. The method according to claim 34, wherein at least one of the zones, selected from heat-up zone, reaction zone and quench zone, has microstructures.

52. The method according to claim 34, wherein, in the quench zone in step iv), the temperature interval ΔT_{K} between reaction temperature T_{R} and T≦120° C. is passed through in the course of a time interval Δt_{K}≦1 s.

53. The method according to claim 34, wherein during the quenching in step iv) a pressure expansion of the reaction mixture proceeds.

54. The method according to claim 34, wherein, subsequently to the quenching, in an additional step v), the reaction mixture is pressure-expanded to ambient pressure.

55. The method according to claim 34, wherein energy integration between the steps ii) and iv) is provided.

56. The method according to claim 34 for producing
dihydroxydioxane from glucose, or
dihydroxydioxane from sucrose, or
furfural from xylose, or
5-hydroxymethylfurfural from fructose.

57. A hydrothermolysis device, comprising
a heat-up zone;
a reaction zone;
a quench zone;
wherein the heat-up zone has a hydraulic diameter of at most 3 mm.

58. The hydrothermolysis device according to claim 57, wherein the reaction zone has a hydraulic diameter of not more than three times the hydraulic diameter of the heat-up zone.

59. The hydrothermolysis device according to claim 57, wherein at least one of the three zones has microstructures.

60. The hydrothermolysis device according to claim 57, comprising
a) a receiver vessel in which an aqueous solution is provided which comprises at least one mono- and/or oligosaccharide;
b) a heat-up zone in which the aqueous solution is heated abruptly;
c) a reaction zone in which the mono- and/or oligosaccharides present in the aqueous solution are partially or completely hydrothermally reacted;
d) a quench zone in which the reaction mixture is cooled to a temperature below 120° C. in the course of at most 0.1 minute;
e) a pressure expansion in which the reaction mixture is expanded to ambient pressure;
f) a discharge vessel in which the resultant reaction mixture is collected.

61. The hydrothermolysis device according to claim 57, wherein the heat-up zone b) comprises an externally heated tube.

62. The hydrothermolysis device according to claim 61, wherein the heated tube has an internal diameter in the range from 20 μm to 2 mm.

63. The hydrothermolysis device according to claim 61, wherein the heated tube has a ratio of tube length to internal diameter of 10² to 10⁷.

64. The hydrothermolysis device according to claim 57, wherein the heat-up zone b) comprises a channel in a microstructured apparatus.

65. The hydrothermolysis device according to claim 64, wherein the channel has a ratio of length to internal diameter of 10² to 10⁷.

66. The hydrothermolysis device according to claim 57, wherein the heat-up zone b) and the quench zone d) are arranged such that energy integration between b) and d) can be utilized.

67. The hydrothermolysis device according to claim 57, wherein two or all three of the zones heat-up zone, reaction zone and quench zone are constructed so as to be not structurally separated from one another.

68. The hydrothermolysis device according to claim 57, wherein the heat-up zone has a hydraulic diameter of at most 0.3 mm.

69. The method according to claim 34, wherein the method is carried out in a hydrothermolysis device, comprising
a heat-up zone;
a reaction zone;
a quench zone;
wherein the heat-up zone has a hydraulic diameter of at most 3 mm.
